(19) United States
(12) Patent Application Publication
(10) Pub. No.: US 2020/0100733 A1
(43) Pub. Date: Apr. 2, 2020

(54) MAGNETIC RESONANCE TOMOGRAPHY SCANNER, SYSTEM, AND METHOD FOR PREVENTING CROSSTALK INTERFERENCE

(71) Applicant: Siemens Healthcare GmbH, Erlangen (DE)

(72) Inventors: Stephan Biber, Erlangen (DE); David Grodzki, Erlangen (DE)

(21) Appl. No.: 16/585,435

(22) Filed: Sep. 27, 2019

(30) Foreign Application Priority Data
Oct. 2, 2018 (EP) 18198141.6

(51) Int. Cl. A61B 6/03; A61B 6/00; H04L 29/08

(52) U.S. Cl. CPC A61B 6/037; A61B 6/548; H04L 67/125; A61B 6/563; A61B 6/566

(57) ABSTRACT

The disclosure relates to a magnetic resonance tomography scanner, a system including two magnetic resonance tomography scanners, and a method for operation thereof which reduces mutual interference between the magnetic resonance tomography scanners. A first magnetic resonance tomography scanner has a control unit for controlling the image acquisition and an interface connected to the control unit for signal communication purposes. The control unit is configured for synchronizing an image acquisition as a function of a signal received via the interface from a second magnetic resonance tomography scanner.

The present patent document claims the benefit of European Patent Application No. 18198141.6, filed Oct. 2, 2018, which is hereby incorporated by reference.

### TECHNICAL FIELD

The disclosure relates to a magnetic resonance tomography scanner, a system including two magnetic resonance tomography scanners, and a method for operation thereof which reduces mutual interference between magnetic resonance tomography scanners.

### BACKGROUND

Magnetic resonance tomography scanners are imaging devices which, in order to image an examination subject, align nuclear spins of the examination subject by a strong external magnetic field and excite them by a magnetic alternating field for precession around the alignment. The precession or return of the spins from this excited state into a state exhibiting lower energy in turn generates a response in the form of a magnetic alternating field, also referred to as a magnetic resonance signal, which is received via antennas.

A spatial encoding scheme is impressed on the signals with the aid of magnetic gradient fields, the spatial encoding subsequently enabling the received signal to be assigned to a volume element. The received signal is then evaluated, and a three-dimensional imaging representation of the examination subject is provided. The generated representation indicates a spatial density distribution of the spins.

The attenuation between excitation pulse and the magnetic resonance signal transmitted by the patient or a specimen amounts to more than 100 dB. As a result, it happens that in spite of radiofrequency shielding cabins serving to enclose magnetic resonance tomography scanners operating in neighboring rooms, excitation pulses of one system in each case interfere with the simultaneous acquisition of a magnetic resonance signal in the adjacent magnetic resonance tomography scanner. Interference events of this type may not be detected due to the irregularity of their occurrence and are assumed to be sporadic disturbances.

### SUMMARY AND DESCRIPTION

It is an object of the present disclosure to provide a magnetic resonance tomography scanner, a system including magnetic resonance tomography scanners, and a method for operating the same which reduces interference events of the type.

The scope of the present disclosure is defined solely by the appended claims and is not affected to any degree by the statements within this summary. The present embodiments may obviate one or more of the drawbacks or limitations in the related art.

The magnetic resonance tomography scanner includes a control unit for controlling the image acquisition, which control unit may influence in particular image acquisition parameters, such as time of the excitation pulse and/or time of the recording of the magnetic resonance signals or also frequency of the excitation pulse and/or frequency range of the receiver during the reception of the magnetic resonance signal. In addition, the magnetic resonance tomography scanner has an interface connected to the control unit for signal communication purposes. As explained below, the interface may be an interface for exchanging data with other magnetic resonance systems or even a radiofrequency interface. The control unit is configured for synchronizing an image acquisition as a function of a signal received via the interface from another magnetic resonance tomography scanner. What is regarded as synchronization in this context is any activity which reduces mutual interference. This may include a coordination with respect to time, but also, for example, a change of frequencies.

In a magnetic resonance tomography scanner, the control unit may also be configured for sending a signal containing information about an upcoming image acquisition to another magnetic resonance tomography scanner. Comparable to a plug-and-socket combination, this feature supplements the preceding magnetic resonance tomography scanner, which is configured for receiving a signal from another magnetic resonance tomography scanner. The information may relate to a time of transmission and/or frequency of an excitation pulse. In certain examples, the magnetic resonance tomography scanner may be configured both for sending and for receiving a signal. The magnetic resonance tomography scanner is also referred to below as the first magnetic resonance tomography scanner.

A system, according to the disclosure, includes a first magnetic resonance tomography scanner and a second magnetic resonance tomography scanner. The first magnetic resonance tomography scanner and the second magnetic resonance tomography scanner each have an interface and a control unit. The first magnetic resonance tomography scanner and the second magnetic resonance tomography scanner are connected via the interfaces for signal communication purposes. The signal communication connection enables at least the transmission of a signal from the first magnetic resonance tomography scanner to the second magnetic resonance tomography scanner, though a bidirectional exchange of information is also conceivable. Point-to-point connections over electrical, optical, or wireless paths are conceivable. Networks such as LANs, WANs, in particular TCP/IP, are also possible as connections for signal communication. The control unit of the first magnetic resonance tomography scanner is configured for sending information relating to an upcoming image acquisition process via the interface to the second magnetic resonance tomography scanner. A time of a planned transmission of an excitation pulse specified in absolute time or relative to the signal, or also to the frequency or a frequency range of the excitation pulse, is conceivable. The control unit of the second magnetic resonance tomography scanner is in this case configured for receiving the information via the interface and for performing an image acquisition as a function of the received information. For example, the controller of the second magnetic resonance tomography scanner may be configured to postpone a magnetic resonance signal acquisition, (e.g., a sequence or a part thereof), so that the excitation pulse of the first magnetic resonance tomography scanner causes no interference. The second magnetic resonance tomography scanner is also referred to below as the other magnetic resonance tomography scanner.

The method is provided for operating a first magnetic resonance tomography scanner having a control unit for controlling the image acquisition and an interface connected to the control unit for signal communication purposes. The method includes the act whereby a signal is received by the control unit via the interface from a second magnetic resonance tomography scanner. This may involve a targeted exchange of information in which the first magnetic resonance tomography scanner receives information or a message containing parameters such as time and/or frequency of an intended image acquisition from the second magnetic resonance tomography scanner via a data interface. It is also conceivable that the first magnetic resonance tomography scanner monitors the environment, for example, via a receiver for magnetic resonance signals.

In another act, the control unit of the first magnetic resonance tomography scanner sets an image acquisition parameter as a function of the received signal. It is conceivable, for example, that a sequence is deferred in time.

In a further act, an image acquisition is performed by the first magnetic resonance tomography scanner in accordance with the set parameter. For example, the sequence may be started at a changed time so that the excitation pulses of both magnetic resonance tomography scanners are applied simultaneously or the excitation pulse of the first magnetic resonance tomography scanner is applied at a time at which the second magnetic resonance tomography scanner is receiving no image-relevant magnetic resonance signals.

In a possible act, the controller determines an image from the received magnetic resonance signals and outputs the image on a display.

Advantageously, the magnetic resonance tomography scanner, the system includes two magnetic resonance scanners and the method for operation thereof enable mutual interference events occurring between two concurrently operating magnetic resonance tomography scanners to be reduced.

In a possible embodiment variant of the magnetic resonance tomography scanner, the interface is configured for exchanging data. In other words, the magnetic resonance tomography scanner is configured both for sending data via the interface to another magnetic resonance tomography scanner and for receiving data from another magnetic resonance tomography scanner via the interface. In this case, the control unit is configured for synchronizing an image acquisition with another magnetic resonance tomography scanner by information exchange via the interface. In other words, the control units coordinate with each other by messages to reach agreement on how the image acquisition will be accomplished so that mutual interference events are reduced.

In a conceivable embodiment variant of the magnetic resonance tomography scanner, the signal includes information relating to a time and/or frequency of a transmit process. In this case, the time may be specified as an absolute value or relative to the time of the sending of the message. A center frequency and/or a bandwidth, a frequency range or also a channel specification that codes a frequency range may be specified as the frequency.

In a possible embodiment variant of the method for operating a first magnetic resonance tomography scanner and a second magnetic resonance tomography scanner, the method additionally includes the act of ascertaining information about an upcoming image acquisition of the second magnetic resonance tomography scanner by a control unit of the second magnetic resonance tomography scanner and, in a further act, sending a signal containing the information to the first magnetic resonance tomography scanner.

For example, the control unit may receive a message via the interface from a second magnetic resonance tomography scanner to the effect that in two seconds the latter will send an excitation pulse at a center frequency equal to the Larmor frequency+100 kHz at a bandwidth of 200 kHz. The first magnetic resonance tomography scanner may then interrupt a sequence prior to an excitation pulse of its own and resume after the second magnetic resonance tomography scanner has terminated its sequence, or send the next excitation pulse simultaneously with the excitation pulse of the second magnetic resonance tomography scanner. In this way, the reciprocal interference caused by an excitation pulse of the second magnetic resonance tomography scanner during a receive phase may advantageously be avoided.

In a possible embodiment variant of the magnetic resonance tomography scanner, the signal includes information relating to a time and/or frequency of a receive process. The information may specify a start and a duration and frequency, such as starting in one second, the second magnetic resonance tomography scanner will, for a duration of two seconds, receive on a center frequency equal to the Larmor frequency minus 300 kHz at a bandwidth of 200 kHz. The first magnetic resonance tomography scanner may then interrupt a sequence in such a way that during this time it will not send an excitation pulse in the cited frequency band.

Advantageously, even during reception of a message relating to a planned reception in other units, a transmission of the first magnetic resonance tomography scanner may be delayed so as to reduce a disturbance.

In a possible embodiment variant, it is conceivable that the control unit of the magnetic resonance tomography scanner is configured for changing the frequency of an image acquisition process as a function of the received information. For example, it is conceivable that an image acquisition includes different slices, these being differentiated by a gradient magnetic field in the z-axis and thus in the effective Larmor frequency of the magnetic resonance signal. In this way, a simultaneous operation with reduced interaction is possible provided the ordering of the slices during the image acquisition is organized in such a way that an acquisition in the same frequency range never occurs simultaneously in the two systems.

The differentiation by way of the frequency advantageously enables a simultaneous acquisition of magnetic resonance signals and consequently a better utilization of the examination time.

In a conceivable embodiment variant of the magnetic resonance tomography scanner, the first magnetic resonance tomography scanner has a receiver as interface. What is regarded as a receiver in this case may be a receiver for magnetic resonance signals including an antenna such as a local coil or body coil. The first magnetic resonance tomography scanner is configured here for detecting an excitation pulse of a second magnetic resonance tomography scanner outside of an image acquisition and for performing the image acquisition as a function of the detected excitation pulse. It is conceivable, for example, that the first magnetic resonance tomography scanner then initially records magnetic resonance signals of a slice at a different effective Larmor frequency or waits until a maximum duration for an acquisition of magnetic resonance signals in the other, second magnetic resonance tomography scanner transmitting the excitation pulse has elapsed.

The synchronization may thus advantageously take place also without a data connection or changes to the workflow in the second magnetic resonance tomography scanner.

In a possible embodiment variant of the method for operating a first magnetic resonance tomography scanner and a second magnetic resonance tomography scanner, the method additionally includes the act of ascertaining information about an upcoming image acquisition of the second magnetic resonance tomography scanner by a control unit of the second magnetic resonance tomography scanner and, in a further act, sending a signal containing the information to the first magnetic resonance tomography scanner.

It is also conceivable in principle for the different measures described to be combined with one another. A protocol for an information exchange in two magnetic resonance tomography scanners may also be provided, thereby enabling the image acquisitions of both systems to be interleaved with one another in an optimized manner so that the acquisition duration changes only insignificantly, without mutual interference occurring. In the simplest case, for example, all of the excitation pulses may be transmitted simultaneously for this purpose, as long as an image acquisition takes place in the same time period in both magnetic resonance tomography scanners.

### BRIEF DESCRIPTION OF THE DRAWINGS

The above-described characteristics, features, and advantages of the disclosure, as well as the manner in which these are realized, will become clearer and more readily understandable in connection with the following description of the exemplary embodiments, which are explained in more detail in conjunction with the drawings.

FIG. 1 depicts a schematic representation of an example of a system including a magnetic resonance tomography scanner, a receive antenna, and a connecting lead.

FIG. 2 depicts a schematic representation of an example of a system including a plurality of magnetic resonance tomography scanners.

FIG. 3 depicts a schematic representation of a flowchart of an embodiment variant of the method.

### DETAILED DESCRIPTION

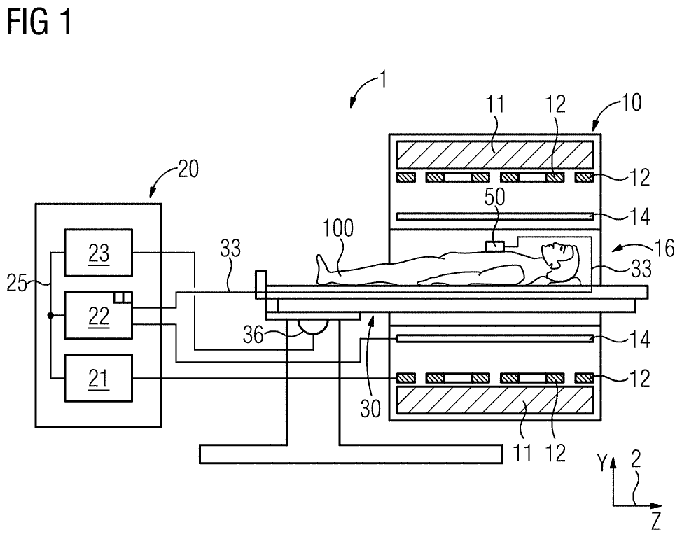

FIG. 1 depicts a schematic representation of an embodiment variant of a magnetic resonance tomography scanner 1 having a local coil 50.

The magnet unit 10 has a field magnet 11 which generates a static magnetic field BO for aligning nuclear spins of samples or of the patient 100 in an acquisition zone. The acquisition zone is characterized by an extremely homogeneous static magnetic field BO, the homogeneity relating in particular to the magnetic field strength or the absolute value. The acquisition zone is virtually spherical in shape and is arranged in a patient tunnel 16 which extends in a longitudinal direction 2 through the magnet unit 10. A patient couch 30 is movable in the patient tunnel 16 by the positioning unit 36. The field magnet 11 may be a superconducting magnet which is able to provide magnetic fields having a magnetic flux density of up to 3T, even more in more recent devices. For lower field strengths, however, it is also possible to utilize permanent magnets or electromagnets having normally conductive coils.

In addition, the magnet unit 10 has gradient coils 12 which are configured to overlay the magnetic field BO with variable magnetic fields in three spatial directions for the purpose of spatially differentiating the acquired imaging regions in the examination volume. The gradient coils 12 may be coils made from conductive wires which may generate fields orthogonal to one another in the examination volume.

The magnet unit 10 also includes a body coil 14 which is configured to radiate a radiofrequency signal supplied by way of a signal line into the examination volume and to receive resonance signals emitted by the patient 100 and output the signals by way of a signal line.

A control unit 20 supplies the magnet unit 10 with the different signals for the gradient coils 12 and the body coil 14 and evaluates the received signals.

Accordingly, the control unit 20 includes a gradient controller 21 configured to supply the gradient coils 12 via supply lines with variable currents which provide the desired gradient fields in the examination volume in a time-coordinated manner.

In addition, the control unit 20 includes a radiofrequency unit 22 configured to generate a radiofrequency pulse having a predefined time characteristic, amplitude and spectral power distribution for exciting a magnetic resonance of the nuclear spins in the patient 100. Pulse powers in the kilowatt range may be achieved in this case. The excitation pulses may be radiated into the patient 100 by way of the body coil 14 or also by way of a local transmit antenna.

A controller 23 communicates with the gradient controller 21 and the radiofrequency unit 22 via a signal bus 25.

The control unit 20 of the magnetic resonance tomography scanner is provided in at least one embodiment variant with a LAN interface 26, via which a communication may take place with other computers and in particular another, second magnetic resonance tomography scanner 101.

A local coil 50 is arranged on the patient 100 and is connected to the radiofrequency unit 22 and its receiver by way of a connecting lead 33.

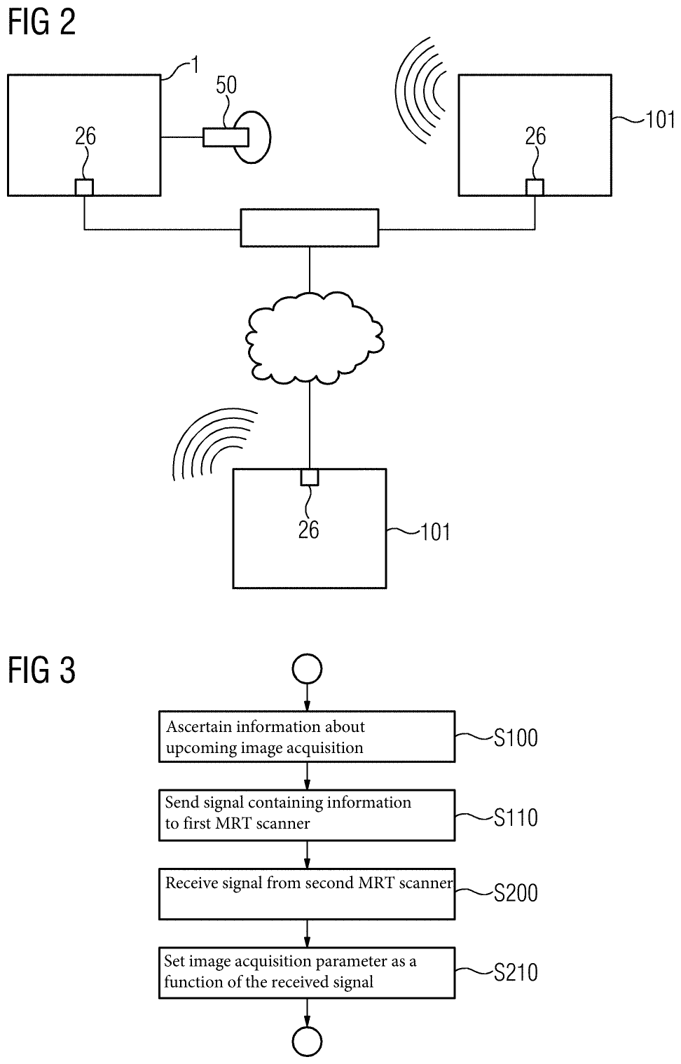

FIG. 2 depicts a system including a plurality of magnetic resonance tomography scanners 1. In this arrangement, the control unit 20 of one magnetic resonance tomography scanner 1 receives a signal from a second magnetic resonance tomography scanner 101 via an interface. The control unit 21 is in this case configured for synchronizing an image acquisition as a function of a signal received via the interface from a second magnetic resonance tomography scanner 101.

A plurality of possible embodiment variants is shown simultaneously in FIG. 2. The interface may on the one hand be the LAN interface 26 via which the magnetic resonance tomography scanner 1 is connected for signal communication purposes to a second magnetic resonance tomography scanner 101. On the other hand, all other interfaces for exchanging information, such as WIFI, WAN or serial or parallel point-to-point connections, are also conceivable.

In one embodiment variant, it is conceivable here that by the signal the other magnetic resonance tomography scanner 101 sends a message about a planned image acquisition. The message may specify that an excitation pulse of duration d will be sent at a defined time instant t on the frequency f by the other magnetic resonance tomography scanner 101. The control unit 20 of the first magnetic resonance tomography scanner 1 then synchronizes its own image acquisition as a function of the information.

One possibility is that the control unit 20 synchronizes an excitation pulse of its own in such a way that it takes place at the same time because, due to the extremely high field strengths necessary for the excitation, the excitation pulses of neighboring second magnetic resonance tomography scanners 101 do not interfere with one another owing to the attenuation already provided by the construction of the magnetic resonance tomography scanners 1, 101.

In contrast, the reception of magnetic resonance signals from the examination volume or patient 100 is more sensitive to disturbances. Because, in this case, an attenuation with respect to the excitation pulse of over 100 dB is present, an excitation pulse of a neighboring second magnetic resonance tomography scanner 101 may disrupt the reception of an MR signal even when there is shielding present. The control unit 20 of the first magnetic resonance tomography scanner 1 may therefore plan and perform the image acquisition in such a way that this does not coincide with the excitation pulse of the second magnetic resonance tomography scanner 101. For example, its own excitation pulses and the readout sequences dependent thereon may be timed such that the receive time windows of the first magnetic resonance tomography scanner 1 do not coincide with the excitation pulses of the second magnetic resonance tomography scanner 101.

It is in this case also conversely possible for the second magnetic resonance tomography scanner 101 to send information about a planned reception. The message may specify that, at a defined time instant t, a magnetic resonance signal is to be recorded by the second magnetic resonance tomography scanner 101 on the frequency f for the duration d. The first magnetic resonance tomography scanner 1 may then set a transmit process of its own such that no transmit process takes place in the time window specified in the message, at least not on a frequency band which includes the frequency f including a bandwidth specified in the message.

Finally, combined messages are furthermore conceivable in which transmit and receive processes are coordinated in an alternating manner between the first magnetic resonance tomography scanner 1 and the second magnetic resonance tomography scanner 101, e.g., in such a way that the imaging devices may perform the image acquisitions with minimum possible delay by interleaving.

In another embodiment variant shown in FIG. 2, it is however also conceivable that the signal is a radio wave of an excitation pulse itself and the interface, for example, the local coil 50 with the radiofrequency unit 22. In this case, a reception may take place also or specifically at times in which the first magnetic resonance tomography scanner 1 itself is recording no MR signal. The control unit 20 is able to detect, based on the excitation pulse, that a second magnetic resonance tomography scanner 101 has just sent an excitation pulse and therefore subsequently plans to record a magnetic resonance signal. It is conceivable in that case, for example, that the first magnetic resonance tomography scanner 1 will then itself transmit no excitation pulses for a certain time. It would also be possible for the first magnetic resonance tomography scanner 1 to use the excitation pulse of the second magnetic resonance tomography scanner itself as a trigger pulse and virtually synchronously transmit an excitation pulse of its own, because there may be pauses without reception, and consequently potential reciprocal interference, between excitation pulse and reception of the magnetic resonance signal.

Irrespective of whether the transmission of an excitation pulse is detected directly by way of the received electromagnetic field of the pulse or recognized by way of a message via the data interface, it is also conceivable in this case that the control unit 20 changes the frequency of the next excitation pulse as a function of the signal. In magnetic resonance tomography, individual slices are differentiated in terms of frequency, and therefore distinguishable, along the direction of the BO field, (e.g., along the z-axis 2), by a superimposed gradient field in the z-direction. The control unit 20 may change the order in which individual slices are sampled so that the first magnetic resonance tomography scanner 1 and the second magnetic resonance tomography scanner 101 in each case acquire slices at a different center frequency and in this way, by the different frequencies, crosstalk, or an interaction is avoided. In this case, an additional degree of freedom which the control unit 20 may use is also the position of the patient 100 on the movable patient couch 30 relative to the isocenter of the field magnet 10. Repositioning the patient 100 a short distance along the z-axis also results in a change in the Larmor frequency for a slice due to the different position in relation to the z-gradient field. The first magnetic resonance tomography scanner 1 may therefore also record the same slice in the body of the patient 100 at different frequencies by a relative movement of the patient along the z-axis such that an interaction with the second magnetic resonance tomography scanner 101 may be avoided.

FIG. 3 depicts a schematic flowchart of a method.

In act S200, the control unit 20 receives a signal via the interface from a second magnetic resonance tomography scanner 101. In this case, the interface may be a LAN interface 26, a WAN interface, or a point-to-point data interface. The interface may be configured for receiving data from the other magnetic resonance tomography scanner 101 including information relating to an image acquisition process of the other magnetic resonance tomography scanner 101. The data may relate to a transmit process of an excitation pulse and/or a receive process of MR signals. The data may include information relating to frequency and time, for example, center frequency, bandwidth, start time, and/or duration.

In one embodiment variant, it is however also conceivable that the signal is an electromagnetic field of the excitation pulse itself and the interface an antenna such as the local coil 50 and the radiofrequency unit 22 for receiving MR signals. In this case, in the interval between its own excitation pulses and MR signal acquisitions, the radiofrequency unit 22 may monitor the frequency band in which the first magnetic resonance tomography scanner 1 operates. With reception of an excitation pulse that is characterized by frequency, duration and/or waveform, the control unit 20 may detect an activity of the second magnetic resonance tomography scanner 101.

In a further act S210, the control unit 20 sets an image acquisition parameter as a function of the received signal. For example, the control unit 20 may delay an image acquisition sequence of its own such that it is not disrupted by the excitation pulse of the second magnetic resonance tomography scanner 101. It is also conceivable to delay an excitation pulse of its own such that it does not interfere with a sequence of the second magnetic resonance tomography scanner 101, for example, in that the excitation pulse is transmitted simultaneously or else so late that it does not disrupt the acquisition of the magnetic resonance signals. By exchanging data relating to the sequence between the two magnetic resonance tomography scanners 1, 101, it is also possible to interleave the sequences in such a way that a minimum of delay is achieved for both sequences.

As well as a variation with respect to time, the frequency is also a conceivable parameter which may be varied by the control unit 20. During a sampling of a plurality of slices along the z-axis, the spatial differentiation is achieved by the gradient of the magnetic field along the z-axis and thereby resulting different Larmor frequencies. By changing the order of the slices to be acquired it is possible, for example, to avoid the simultaneous use of the same frequency. Even in the case of the unchanged slice, a different frequency may be chosen via a change in the position of the patient couch 30 along the z-axis and in this way a collision may be avoided.

An image acquisition in accordance with the set parameter(s) is then performed by the magnetic resonance tomography scanner 1 under the control of the control unit 20. Finally, the image may be output on a display.

In a possible embodiment variant of the method, information about an upcoming image acquisition by the second magnetic resonance tomography scanner 101 is ascertained in act S100 by the control unit 20 of the second magnetic resonance tomography scanner 101. For example, the control unit 20 ascertains one or more items of information from the following list: frequency of an excitation pulse, frequency distribution of an excitation pulse, start time for the transmission of the excitation pulse, duration of the excitation pulse, frequency of one or more MR signal acquisitions, frequency distribution of the MR signal acquisition(s), start time, and duration of the MR signal acquisition(s).

In a further act S110, the second magnetic resonance tomography scanner 101 sends a signal containing the information to the first magnetic resonance tomography scanner 1. This may be accomplished, for example, via the LAN interfaces 26 and a data network connected thereto.

The method may in this regard also be supplemented by further acts in which the coordination of the image acquisition is agreed in detail between the two magnetic resonance tomography scanners. For example, the use of different frequencies at a defined time instant may be agreed by an adjustment of the order in which slices are sampled. It is also conceivable that the sending of radiofrequency pulses is set in such a way that both magnetic resonance tomography scanners 1, 101 transmit simultaneously, because interference events occur predominantly during the reception of MR signals in the first magnetic resonance tomography scanner 1 when the second magnetic resonance tomography scanner 101 is transmitting. In addition, further measures to coordinate transmit and receive activities are possible, for example, by pauses in reception that result due to gradient switchovers.

Although the disclosure has been illustrated and described in greater detail on the basis of the exemplary embodiments, the disclosure is not limited by the disclosed examples and other variations may be derived herefrom by the person skilled in the art without leaving the scope of protection of the disclosure. It is therefore intended that the foregoing description be regarded as illustrative rather than limiting, and that it be understood that all equivalents and/or combinations of embodiments are intended to be included in this description.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

1. A magnetic resonance tomography scanner comprising:
a control unit for controlling an image acquisition; and
an interface connected to the control unit for signal communication,
wherein the control unit is configured for synchronizing the image acquisition as a function of a signal received via the interface from a second magnetic resonance tomography scanner.

2. The magnetic resonance tomography scanner of claim 1, wherein the interface is configured for exchanging data, and
wherein the control unit is configured for synchronizing the image acquisition with a second magnetic resonance tomography scanner by an information exchange via the interface.

3. The magnetic resonance tomography scanner of claim 1, wherein the interface is a receiver, and
wherein the magnetic resonance tomography scanner is configured for detecting an excitation pulse of the second magnetic resonance tomography scanner outside of an image acquisition and for performing the image acquisition as a function of the detected excitation pulse.

4. A magnetic resonance tomography scanner comprising:
a control unit for controlling the image acquisition; and
an interface connected to the control unit for signal communication,
wherein the control unit is configured for sending a signal containing information about an upcoming image acquisition to a second magnetic resonance tomography scanner.

5. The magnetic resonance tomography scanner of claim 4, wherein the interface is configured for exchanging data, and
wherein the control unit is configured for synchronizing the image acquisition with a second magnetic resonance tomography scanner by an information exchange via the interface.

6. The magnetic resonance tomography scanner of claim 5, wherein the signal includes information relating to a time, a frequency, or both a time and a frequency of a transmit process.

7. The magnetic resonance tomography scanner of claim 6, wherein the signal includes information relating to a time, a frequency, or both a time and a frequency of a receive process.

8. The magnetic resonance tomography scanner of claim 7, wherein the control unit is configured for changing the frequency of an image acquisition process as a function of the received information.

9. The magnetic resonance tomography scanner of claim 4, wherein the signal includes information relating to a time, a frequency, or both a time and a frequency of a transmit process.

10. The magnetic resonance tomography scanner of claim 9, wherein the signal includes information relating to a time, a frequency, or both a time and a frequency of a receive process.

11. The magnetic resonance tomography scanner of claim 10, wherein the control unit is configured for changing the frequency of an image acquisition process as a function of the received information.

12. The magnetic resonance tomography scanner of claim 4, wherein the signal includes information relating to a time, a frequency, or both a time and a frequency of a receive process.

13. The magnetic resonance tomography scanner of claim 12, wherein the control unit is configured for changing the frequency of an image acquisition process as a function of the received information.

14. A system comprising:
a first magnetic resonance tomography scanner having a control unit and an interface; and
a second magnetic resonance tomography scanner having a control unit and an interface,
wherein the interface of the first magnetic resonance tomography scanner is connected to the interface of the second magnetic resonance tomography scanner via the for signal communication purposes,
wherein the control unit of the first magnetic resonance tomography scanner is configured for sending information relating to an upcoming image acquisition process via the interface of the first magnetic resonance tomography scanner, and
wherein the control unit of the second magnetic resonance tomography scanner is configured for receiving the information via the interface of the second magnetic resonance tomography scanner and for performing an image acquisition as a function of the received information.

15. A method for operating a first magnetic resonance tomography scanner having a control unit for controlling the image acquisition and an interface connected to the control unit for signal communication, the method comprising:
receiving a signal by the control unit via the interface from a second magnetic resonance tomography scanner;
setting an image acquisition parameter as a function of the received signal by the control unit; and
performing an image acquisition in accordance with the set parameter.

16. The method of claim 15, further comprising:
ascertaining information about an upcoming image acquisition of the second magnetic resonance tomography scanner by a control unit of the second magnetic resonance tomography scanner; and
sending a signal containing the information to the first magnetic resonance tomography scanner.

17. A computer-readable storage medium on which is stored electronically readable control information, wherein, the electronically readable control information, when executed by a control unit of the first magnetic resonance tomography scanner and/or second magnetic resonance tomography scanner, is configured to cause the control unit to:
receive a signal via the interface from a second magnetic resonance tomography scanner;
set an image acquisition parameter as a function of the received signal; and
perform an image acquisition in accordance with the set parameter.
